(19) United States
(12) Patent Application Publication
(10) Pub. No.: US 2026/0137611 A1
(43) Pub. Date: May 21, 2026

(54) NOVEL COMPOSITIONS

(71) Applicant: DSM IP Assets B.V., Maastricht (NL)

(72) Inventor: Christine MENDROK-EDINGER, Kaiseraugst (CH)

(21) Appl. No.: 19/121,564

(22) PCT Filed: Oct. 27, 2023
(86) PCT No.: PCT/EP2023/080043

§ 371 Date: Apr. 16, 2025

(30) Foreign Application Priority Data
Oct. 28, 2022 (EP) 22204297.0

(51) Int. Cl. A61K 8/60; A61K 8/06; A61K 8/27; A61K 8/29; A61K 8/35; A61K 8/37; A61K 8/49; A61K 8/89; A61Q 17/04

(52) U.S. Cl. CPC A61K 8/60; A61K 8/064; A61K 8/27; A61K 8/29; A61K 8/35; A61K 8/37; A61K 8/4946; A61K 8/4966; A61K 8/89; A61Q 17/04

(57) ABSTRACT

The present invention relates to emulsions containing at least one sunscreen active agent and having improved sun protection factor (SPF) properties, as well as methods of improving sun protection factor (SPF) properties.

The present invention relates to compositions containing at least two different inorganic sunscreen active agents and having improved sun protection factor (SPF) properties, as well as methods of improving sun protection factor (SPF) properties.

The use of compositions containing sunscreen active agents (also called sunscreens) is very common nowadays. The sunscreen active agents in such compositions protect keratinous material such as in particular the skin from the harm caused by UV radiation, including harm from both UVA and UVB rays, which are known to not only contribute to skin wrinkling but also to foster the development of skin diseases, such as lupus erythematosus and melanoma and non-melanoma skin cancer. Thus, to get ultimate skin protection, there's a constantly increasing need for sunscreens exhibiting high SPF's (Sun Protection Factor).

Inorganic sunscreen active agents are popular ingredients to be used in sunscreens for achieving safe and highly efficient sunscreens that offer both good stability and excellent skin compatibility.

A standard measure for determining the amount of protection a composition containing sunscreen active agent provides against UV radiation is the sun protection factor (SPF). However, given the finite amount of approved sunscreen active agents worldwide, increasing SPF of compositions containing sunscreen active agents can be difficult.

Thus, there is an ongoing need for preferably natural ingredients which are able to increase the SPF of inorganic sunscreen active ingredients in compositions.

Surprisingly it has now been found that human milk oligosaccharides are able to significantly increase the SPF of a composition comprising at least two different inorganic UV-filters selected from the group of zinc oxide and titanium dioxide.

Thus, in a first embodiment, the present invention relates to a composition for keratinous materials (for example, hair or skin) comprising at least two different inorganic UV-filters selected from the group of zinc oxide and titanium dioxide and at least one human milk oligosaccharide. Preferably, the composition is in the form of a water in oil emulsion (W/O emulsion).

The present invention also relates to methods of increasing the sun protection factor (SPF) properties of a composition comprising at least two different inorganic UV-filters selected from the group of zinc oxide and titanium dioxide, said method comprising adding at least one human milk oligosaccharide to the composition in an amount effective to increase the SPF properties of the composition.

The invention also relates to a use of a human milk oligosaccharide for increasing the sun protection factor (SPF) of a composition comprising at least two different inorganic UV-filters selected from the group of zinc oxide and titanium dioxide.

The present invention also relates to methods of preparing compositions having improved sun protection factor (SPF) properties comprising adding at least two different inorganic UV-filters selected from the group of zinc oxide and titanium dioxide and at least one human milk oligosaccharide to the composition during preparation of the composition. It is well understood that said improved properties are to be compared to a composition not comprising the human milk oligosaccharide with all the definitions and preferences as given herein.

The present invention further relates to methods of making a composition, said method comprising combining at least two different inorganic UV-filters selected from the group of zinc oxide and titanium dioxide and at least one human milk oligosaccharide in said composition.

In accordance with the invention is also the use of the composition of the invention for protection of skin from the effects skin aging (especially for protection from UV-induced skin aging) such as in particular wrinkles and as a sun protection composition.

Finally, a subject-matter of the invention is a method for the cosmetic treatment of keratinous substances such as in particular the skin, wherein a composition as defined herein is applied to the said keratinous substances such as in particular to the skin. The method is in particular suitable to protect the skin against the adverse effects of UV-radiation such as in particular sun-burn and/or photoageing.

The term ‘keratinous materials’ means the skin (body, face, contour of the eyes, scalp), head hair, eyelashes, eyebrows, bodily hairs, nails and/or lips. Preferably, in all embodiments the keratinous material is the skin.

The term ‘human milk oligosaccharides’ (HMOs) refers to a family of structurally diverse unconjugated glycans that are highly abundant in and unique to human milk. Originally, HMOs were proposed to be prebiotic “bifidus factors,” or human milk glycans found to promote growth in Bifidobacterial species of the gut and found uniquely in the stool of breast-fed infants compared to formula fed infants.

The SPF according to the present invention is understood to be measured by the in vitro SPF method as outlined in ISO 24443 (Determination of sunscreen UVA photoprotection in vitro, Jan. 6, 2012) and as exemplified in the examples.

HMOs are generally composed of the five monosaccharides glucose (Glc), galactose (Gal), N-acetylglucosamine (GlcNAc), fucose (Fuc) and sialic acid (Sia), with N-acetylneuraminic acid (Neu5Ac) as the predominant if not only form of Sia. More than two hundred different HMOs have been identified so far. The most important ones are 2′-fucosyllactose (2′FL), lacto-N-neotetraose (LNnT), 3-fucosyllactose (3FL), difucosyllactose (DFL), Lacto-N-fucopentaose I (LNFP I), 3′Sialyllactose Sodium Salt (3′SL), 6′Sialyllactose Sodium Salt (6′SL), and Lacto-N-Tetraose (LNT).

HMOs can be isolated from breast milk, or they can be produced chemically or biochemically. HMOs are available commercially from a variety of producers.

For the purpose of the present invention the source of the HMO is not essential. It is clear that HMOs from different sources can be used.

Particularly suitable HMO's in all embodiments of the present invention are fucosylated HMO's such as in particular α1-2 respectively α1-3 fucosylated HMO's; sialylated HMO's such as in particular sialyllactoses, as well as the neutral core HMO's as well as any mixtures thereof.

Particularly preferred fucosylated HMO's according to the present invention are 2′-fucosyllactose (2′FL; CAS No: 41263-94-9), 3-fucosyllactose (3FL; CAS No: 41312-47-4), difucosyllactose (DFL; also known as Lactodifucotetraose; CAS No: 20768-11-0) and Lacto-N-fucopentaose I (LNFP-I; CAS No: 7578-25-8).

Particularly preferred sialylated HMO's according to the present invention are sialyllactoses as well as salts thereof (preferably the sodium salts) such as in particular 3′sialyllactose (3′SL), 6′sialyllactose (6′SL) as well as the respective sodium salts thereof (CAS No's: 35890-39-2 (3′sialyllactose); 128596-80-5 (3′sialyllactose sodium salt); 35890-39-2 (6′sialyllactose); 157574-76-0 (6′sialyllactose sodium salt)).

Particularly preferred neutral core HMO's are built up from galactose, N-acetylglucosamine and glucose monosaccharide units, preferably linked through β-(1-3) or β-(1-4) bonds with each other. Preferably, in all embodiments herein, the galactose is D-galactose and the glucose is D-glucose.

Particularly preferred neutral core HMO's, in all embodiments of the present invention are lacto-N-triose II, LNT, LNnT, LNH, LNnH, pLNH and pLNnH as well as mixtures thereof are used. Most preferred neutral core HMO's in all embodiments of the present invention are Lacto-N-(neo)tetraoses, i.e. lacto-N-tetraose (CAS No: 14116-68-8) and/or lacto-N-neotetraose (CAS No: 13007-32-4).

The total amount of the HMO(s) according to the present invention in the compositions according to the present invention is preferably at least 0.01 wt.-%, more preferably at least 0.1 wt.-%, even more preferably at least 0.2 wt.-%, such as at least 0.3 wt.-% or at least 0.5 wt.-%, based on the total weight of the composition. In particular the total amount of the at least one HMO(s) according to the present invention in the compositions according to the present invention is preferably selected in the range from 0.01 to 10 wt.-%, more preferably in the range from 0.1 to 7.5 wt.-%, most preferably in the range from 0.2 to 5 wt.-%, based on the total weight of the composition. Further suitable ranges are from 0.25 to 5 wt.-%, from 0.5 to 4 wt.-%, from 0.1 to 3 wt.-%, from 0.25 to 3 wt.-% and from 0.3 to 3 wt.-%. Particularly preferred ranges according to the present invention are from 0.1 to 5 wt.-%, more preferably from 0.25 to 3 wt.-%, such as from 0.25 to 1.5 wt.-% 0.5 to 5 wt.-%, or 0.5 to 4 wt.-%.

Preferably in all embodiments of the present invention the at least one HMO is selected from the group consisting of 2′-fucosyllactose (2′FL), 3-fucosyllactose (3FL), difucosyllactose (DFL), lacto-N-fucopentaose I (LNFP-I), 3′sialyllactose sodium salt (3′SL), 6′sialyllactose sodium salt (6′SL), lacto-N-neotetraose (LNnT), Lacto-N-tetraose (LNT), as well as mixtures thereof, more preferably from 3′Sialyllactose Sodium Salt (3′SL), 3-fucosyllactose (3FL), 6′sialyllactose sodium salt (6′SL), lacto-N-neotetraose (LNnT), Lacto-N-tetraose (LNT), difucosyllactose (DFL) as well as mixtures thereof, most preferably from 2′-fucosyllactose (2′FL) and 6′sialyllactose sodium salt (6′SL), 2′FL being the most preferred.

It is well understood, that the compositions according to the present invention May comprise one or more HMO's. Preferably, however, the compositions according to the present invention comprise one or two, more preferably solely one HMO, such as most preferably selected from the group of 2′FL, 3FL, 3′SL, 6′SL, LNT and/or LNnT. The most preferred HMO in all embodiments of the present invention is 2′FL as it leads to a particular pronounced increase in the SPF.

The term ‘inorganic UV-filter’ as used herein refers to any metal oxide particles having UV-filter properties as defined above and are thus useful for incorporation into compositions as UV filters. Such inorganic UV-filters are well known to a person skilled in the art and are often referred to as micronized or microfine UV filters such as in particular micronized or microfine titanium dioxide and zinc oxide.

In a particular preferred embodiment, the compositions according to the present invention comprise at least one titanium dioxide and at least one zinc oxide UV-filter. In an even more preferred embodiment, the compositions according to the present invention comprises as UV-filters solely inorganic UV-filters, such as in particular at least one titanium dioxide and at least one zinc oxide UV-filter, most preferably one titanium dioxide and one zinc oxide UV-filter.

The particle size of such inorganic UV-filter is not particularly limited. In general, suitable (primary) particle sizes for an efficient UV-light absorption are selected in the range of 2 to 200 nm.

In all embodiments of the present invention, the (total) amount of the inorganic UV-filters in the compositions according to the present invention is preferably selected in the range from 0.5 to 30 wt.-%, more preferably in the range from 1 to 30 wt.-%, and most preferably in the range from 2 to 30 wt.-%, based on the total weight of the composition. Further suitable amounts are selected in the range from 1 to 25 wt.-%, 1 to 20 wt.-%, 1 to 15 wt.-%, 3.0 to 25 wt.-%, 4 to 25 wt.-%, 5 to 20 wt.-% or 10 to 25 wt.-%.

Even more advantageously, the (total) amount of inorganic titanium dioxide UV-filters in the compositions according to the present invention is selected in the range of 0.5 to 15 wt.-%, preferably in the range of 1 to 15 wt.-%, more preferably in the range of 2 to 10 wt.-%, most preferably in the range of 2 to 6 wt.-% or 3 to 6 wt.-%, based on the total weight of the composition.

Even more advantageously, the (total) amount of inorganic zinc oxide UV-filters in the compositions according to the present invention is selected in the range of 0.5 to 25 wt. %, preferably in the range of 1 to 20 wt.-%, more preferably in the range of 2 to 20 wt.-%, most preferably in the range of 5 to 20 wt.-% or 7.5 to 20 wt.-%, based on the total weight of the composition.

Preferably, if present in the compositions according to the present invention the ratio (w/w) of the titanium dioxide UV-filter(s) to the zinc oxide UV-filter(s) is selected in the range from 1:5 to 5:1, preferably from 1:2 to 2:1. Even more preferably the amount of zinc oxide in the compositions according to the present invention is higher than the amount of titanium dioxide such as in the range of 5:1 to 1.25:1, most preferably in the range of 2.5:1 to 1.25:1.

In one particular embodiment it is preferred that the compositions according to the present invention comprise solely inorganic UV filters, such in particular solely Zinc oxide and titanium dioxide UV-filters with all the definitions and preferences as given herein.

According to the invention, it is advantageous that the inorganic UV-filters are surface-coated. The surface coating may comprise providing the metal oxide particles with a thin hydrophilic or hydrophobic inorganic or organic layer by methods known per se. According to the present invention the different surface coatings can also comprise water. As a result of the surface treatment, the metal oxide is given a hydrophilic, amphiphilic or hydrophobic character.

Examples of inorganic surface coatings which are suitable for the purposes of the instant invention comprise aluminum oxide (Al₂O₃), aluminum hydroxide Al(OH)₃, aluminum oxide hydrate (also: Alumina, CAS-No.: 1333-84-2), sodium hexametaphosphate (NaPO₃)₆, sodium meta-phosphate (NaPO₃)ₙ, silicon dioxide (SiO₂) (also: Silica, CAS-No.: 7631-86-9), and iron oxide (Fe₂O₃). These inorganic surface coatings can be present on their own, in combination and/or in combination with organic coating materials, in particular as outlined below.

Examples of organic surface coatings which are suitable for use in the present invention include vegetable or animal aluminum stearate, fatty acids such as stearic acid and lauric acid, dimethylpolysiloxane (also: dimethicone), methylpolysiloxane (methicone), simethicone, trimethoxycaprylylsilane, triethoxycaprylylsilane, octyltrimethoxysilane and cetyl phosphates such as potassium cetyl phosphate as well as any mixtures thereof. These organic surface coatings can be present on their own, in combination and/or in combination with inorganic coating materials.

The crystalline form of the titanium dioxide may be of any crystal or amorphous type. For example, titanium dioxide may be any type of amorphous, rutil, anastase, brookite or a mixture thereof. Preferably, in all embodiments of the present invention, the crystalline form of the titanium dioxide is rutil.

It is furthermore preferred that the zinc oxide is a white powder consisting of zinc oxide present as wurtzite crystal structures.

Titanium dioxide UV-filters for use according to the present invention including any pre-dispersions thereof are e.g. available as PARSOL® TX (INCI: titanium dioxide, silica, dimethicone) at DSM Nutritional Products Ltd., Micro Titanium dioxide MT-01 at Tayca or TTO-551 at Ishihara Sangyo Kaisha. Also suitable is double coated titanium dioxide having an inner alumina coating and an outer simethicone coating e.g. commercially available as Eusolex T-2000 at EMD chemicals Inc./Rona.

Zinc oxide UV-filters for use according to the present invention including any pre-dispersions thereof are e.g. available from BASF as Z-Cote or Z-Cote HP1 (2% dimethicone coating), from Tayca as MZ-505S (5% methicone coating), or from DSM Nutritional Products Ltd as PARSOL® ZX (2-3.5% triethoxycaprylylsilane coating).

In a particular embodiment the compositions according to the present invention comprise as inorganic UV-filters at least one double coated titanium dioxide and at least one coated zinc oxide, most preferably in the absence of any further microfine metal oxides (i.e. one double coated titanium dioxide and one coated zinc oxide).

Most preferably in all embodiments according to the present invention the titanium dioxide UV-filter is either a double coated titanium dioxide, even more preferably a double coated titanium dioxide having an inner silica and an outer organic coating or a silica coated titanium dioxide.

Preferably, the inner silica coating layer of the double coated titanium dioxide consists of a minimum of 0.5 wt.-% of inorganic silica (based on titanium dioxide). More, preferably the inner coating layer consists of 0.5 wt.-% to 50 wt.-%, most preferably of 1 wt.-% to 20 wt.-% of inorganic silica (based on titanium dioxide).

The outer coating of the double coated titanium dioxide is preferably selected from the class of organic coatings such e.g. silicone oils (e.g. simethicones, methicones, dimethicones, polysilicone-15), alkyl silanes (e.g. octyl tri (m) ethoxy silane), olefinic acids (e.g. stearic acid), or polyols (e.g. glycerol) and can be applied to the titanium dioxide particle by methods known to a person skilled in the art e.g. described in FI57124. Preferably the outer coating is selected from the group consisting of simethicone, methicone, dimethicone, polysilicones-15, stearic acid and octyl trimethoxy silane. Most preferably the outer coating is dimethicone. Even more preferably, the amount of the outer coating layer is at least 0.25 wt.-% based on the titanium dioxide. Preferably the amount of the outer coating is selected in the range of 0.5 wt.-% to 50 wt.-%, most preferably in the range of 0.5 wt.-% to 10 wt.-%, based on the titanium dioxide.

In all embodiments of the present invention, the double coated titanium dioxide according to the present invention preferably has a titanium dioxide content selected in the range of 70-95 wt.-% and a silicon dioxide content selected in the range of 5-20 wt.-%, such as preferable a titanium dioxide content selected in the range of 80-90 wt.-% and a silicon dioxide content selected in the range of 10 to 15 wt.-%, with the proviso that the total content of titanium dioxide and silicone dioxide is selected in the range of 90-100 wt.-%.

Preferably, the double coated titanium dioxide has a mean primary particle size in the range from 2 to 100 nm, more preferably in the range of 5 to 50 nm, most preferably in the range of 10 to 25 nm and a secondary particle size between 0.025 and 1 μm, such as preferably between 0.05 and 0.075 μm.

Particularly suitable double coated titanium dioxide according to the present invention contains a rutil-type titanium dioxide (TiO₂) core with a double coating of silica (inner coating) and dimethicone (outer coating) and has titanium dioxide content of at least 75 wt.-%, preferably in the range from 82-87 wt.-% and a silicon dioxide content of at least 10 wt.-%, preferably in the range from 10.5 to 14.5 wt.-%, and a mean particle size distribution Dn50 of 25 to 100 nm, preferably 40 to 80 nm (analysed by Laser diffraction measurements with a Malvern Mastersizer 2000), which double coated titanium dioxide is e.g. commercially available as PARSOL® TX (INCI: titanium dioxide, silica, dimethicone) at DSM nutritional products Ltd.

Most preferably, in all embodiments according to the present invention, the zinc oxide is either uncoated or coated with triethoxycaprylylsilane. Most preferably, the zinc oxide is a white powder consisting of zinc oxide present as wurtzite crystal structures, coated with triethoxycaprylylsilane, which has a zinc oxide content of 96-98%, a triethoxycaprylylsilane content of 2-3.5% and a mean particle size of 90 to 130 nm (analysed by Laser diffraction measurements with a Malvern Mastersizer 2000) which is commercially available as PARSOL® ZX from DSM Nutritional Products Ltd.

If nothing else is stated in this specification any given parts and percentages are per weight and based on the total weight of the composition.

Preferably, the human milk oligosaccharide according to the present invention and inorganic UV-filters selected from the group of zinc oxide and titanium dioxide are present in the compositions and methods of the invention in a weight ratio between 1:50 and 1:1, preferably between 1:40 and 1:7.5, and preferably between 1:30 and 1:5, such as in the range of 1:25 to 1:15. Preferably more inorganic UV-filters selected from the group of zinc oxide and titanium dioxide are present than HMO(s) on a weight basis.

As the compositions according to the invention are intended for topical application, it is well understood that they comprise a physiologically acceptable medium, i.e. a medium compatible with keratinous substances, such as the skin, mucous membranes, and keratinous fibres. In particular, the physiologically acceptable medium is a cosmetically acceptable carrier.

The term ‘cosmetically acceptable carrier’ as used herein refers to all carriers and/or excipients and/or diluents conventionally used in topical cosmetic compositions such as in particular in skin care preparations.

The exact amount of carrier will depend upon the actual level of the UV filters and any other optional ingredients that one of ordinary skill in the art would classify as distinct from the carrier (e.g., other active ingredients).

In an advantageous embodiment, the compositions according to the present invention comprise from 50% to 99%, preferably from 60% to 98%, more preferably from 70% to 98%, such as in particular from 80% to 95% of a carrier, based on the total weight of the composition.

In a particular advantageous embodiment, the carrier in all embodiments of the present invention consists furthermore of at least 30 wt. %, more preferably of at least 40 wt.-%, most preferably of at least 45 wt.-% of water, such as in particular of 50 to 90 wt.-%, 50 to 80 wt.-%, 50 to 75 wt.-%, 50 to 60 wt.-% of water.

In particular, the compositions according to the present invention are cosmetic or pharmaceutical compositions, preferably cosmetic (non-therapeutic) compositions.

In one embodiment, the compositions according to the present invention are applied to mammalian keratinous tissue such as in particular to human skin or the human scalp and hair.

The term “cosmetic composition” as used in the present application refers to cosmetic compositions as defined under the heading “Kosmetika” in Rompp Lexikon Chemie, 10^{th} edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992.

Preferred compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment, the compositions according to the invention are light-protective preparations (sun care products, sunscreens), such as sun protection milks, sun protection lotions, sun protection creams, sun protection oils, sun blocks or day care creams with a SPF. Of particular interest are sun protection creams, sun protection lotions and sun protection milks.

The compositions of the invention (including the carrier) may comprise conventional adjuvants and additives, such as preservatives/antioxidants, fatty substances/oils, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing agents, as well as agents to improve elasticity and skin barrier and carriers and/or excipients or diluents conventionally used in compositions.

If nothing else is stated, the excipients, additives, diluents, etc. mentioned in the following are suitable for compositions according to the present invention. The necessary amounts of the cosmetic and dermatological adjuvants and additives can, based on the desired product, easily be determined by the skilled person.

The additional ingredients can either be added to the oily phase, the aqueous phase or separately as deemed appropriate. The mode of addition can easily be adapted by a person skilled in the art.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council, without being limited thereto.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

Suitable W/O- or W/Si-emulsifiers according to the present invention are polyglyceryl-2-dipolyhydroxystearate, PEG-30 dipolyhydroxystearate, cetyl dimethicone copolyol, polyglyceryl-3 diisostearate, polyglycerol esters of oleic/isostearic acid, polyglyceryl-6 hexaricinolate, polyglyceryl-4-oleate, oleate/PEG-8 propylene glycol cocoate, magnesium stearate, sodium stearate, potassium laurate, potassium ricinoleate, sodium cocoate, sodium tallowate, potassium castorate, sodium oleate, and mixtures thereof. Further suitable W/Si-emulsifiers are Lauryl Polyglyceryl-3 Polydimethylsiloxyethyl Dimethicone and/or PEG-9 Polydimethylsiloxyethyl Dimethicone and/or Cetyl PEG/PPG-10/1 Dimethicone and/or PEG-12 Dimethicone Crosspolymer and/or PEG/PPG-18/18 Dimethicone. The at least one W/O emulsifier is preferably used in an amount of about 0.001 to 10 wt.-%, more preferably in an amount of 0.2 to 7 wt.-% with respect to the total weigh of the composition.

Further suitable W/O emulsifiers, which can be used in accordance of the present invention are Polyglyceryl-2 Sesquiisostearate, Polyglyceryl-2 Oleate, Polyglyceryl-3 triolivate, Polyglyceryl-3 Diisostearate, Polyglyceryl-4 Isostearate, Polyglyceryl-4 Polycastorate/Hemp Seedate, Polyglyceryl-4 Isostearate, Polyglyceryl-6 Polyricinoleate, Polyglyceryl-10 Decaoleate, Polyglyceryl-10 Pentaoleate, Polyglyceryl-3 Pentaolivate, Polyglyceryl-10 Pentahydroxystearate, Polyglyceryl-10 Heptahydroxystearate, Polyglyceryl-10 Pentaoleate, PEG-5 Hydrogenated Castor Oil PEG-7 Hydrogenated Castor Oil, PEG-30 Dipolyhydroxystearate, PEG-40 Sorbitan Peroleate, PEG-2 Stearate, PEG-2 Oleate, PEG-80 Sorbitan Oleate, PEG-10 Dimethicone, Cetyl PEG/PPG-10/1 Dimethicone, Lauryl PEG-8 Dimethicone, Cetyl PEG/PPG-7/3 Dimethicone, Lauryl PEG-9 Polydimethylsiloxyethyl Dimethicone, Ceteth-2, Sorbeth-20, Sorbitan Olivate, Sorbitan Isostearate, Sorbitan Oleate, Sorbitan Laurate, Sorbitan monostearate, Sorbitan trioleate, Lanolin Alcohol, Lecithin, Glyceryl Stearate, Propylene Glycol Hydroxystearate, Stearalkonium Bentonite, Quaternium-90 Bentonite, Polyoxyethylene Lauryl Ethen Caprylate, Stearyl Alcohol Ether/Fatty Glyceride, Methyl Glucose Sesquiisostearate, Isostearyl Diglyceryl Succinate, Methyl Glucose Dioleate, Methyl Glucose Sesquistearate as well as Oleyl Hydroxyethyl Imidazoline.

Particular suitable W/O emulsifier mixtures to be used in accordance with the invention encompass Polyglyceryl-2 Sesquiisostearate (and) Cera Alba (Beeswax) (and) Cera Microcrystallina (and) Mineral Oil (and) Magnesium Stearate (and) Aluminum Tristearate (Hostacerin WO), Glyceryl Oleate (and) Polyglyceryl-3 Polyricinoleate (and) Olea europaea Oil Unsaponifiables (Plantasens® Emulsifier CP5), Polyglyceryl-3 Polyricinoleate (and) Sorbitan Oleate (Plantasens® Natural Emulsifier CP40), C13-15 Alkane (and) Disteardimonium Hectorite (and) Polyglyceryl-3 Polyricinoleate. (BENTONE® LUXE XO), Caprylic/Capric Triglyceride (and) Stearalkonium Hectorite (and) Polyglyceryl-3 Diisostearate (and) Polyglyceryl-3 Polyricinoleate (BENTONE® LUXE WN), Polyglyceryl-4 Diisostearate/Polyhydroxystearate/Sebacate (and) Caprylic/Capric Triglyceride (and) Polyglyceryl-3 Oleate (and) Diisostearoyl Polyglyceryl-3 Dimer Dilinoleate (ISOLAN® 17 MB); Polyglyceryl-4 Isostearate (and) Cetyl PEG/PPG-10/1 Dimethicone (and) Hexyl Laurate (Silube® PCH); Polyglyceryl-4 Oleate (and) PEG-8 Propylene Glycol Cocoate; Dicocoyl Pentaerythrityl Distearyl Citrate (and) Sorbitan Sesquioleate (and) Cera Alba (and) Aluminum Stearates (DEHYMULS® E); Cyclopentasiloxane (and) PEG-10 Dimethicone (and) Disteardimionium Hectorite (and) Silica Dimethyl Silylate (iEmul WO); Cetyl PEG/PPG-10/1 Dimethicone (and) Bis-PEG/PPG-14/14 Dimethicone (and) Cyclopentasiloxane (SanCos P2); Cyclopentasiloxane (and) PEG/PPG-19/19 Dimethicone (DOWSIL™ BY 11-030); Sorbitan Oleate (and) Hydrogenated Castor Oil (and) Synthetic Beeswax (and) Stearic Acid (and) Palmitic Acid (ERCAMULS LF 81 V); Polyglyceryl-6 Polyhydroxystearate (and) Polyglyceryl-6 Polyricinoleate (and) Polyglyceryl-6 (Emulium® Illustro); Ceteareth-6 (and) Stearyl Alcohol (Feligel 319); Polyglyceryl-2 Oleate, Polyhydroxystearic Acid and Polyglyceryl-2 Stearate (PolyAquol™ OS2)

Suitable W/SI emulsifiers to be used according to the present invention are e.g. Lauryl PEG-9 Polydimethylsiloxyethyl Dimethicone; PEG-8 Dimethicone; Lauryl PEG-10 Tris(trimethylsiloxy)silylethyl Dimethicone; Cetyl Diglyceryl Tris(Trimethylsiloxy) Silylethyl Dimethicone; Lauryl PEG-9 Polydimethylsiloxyethyl Dimethicone; PEG-9 Dimethicone; PEG-10 Dimethicone; Lauryl PEG-9 Polydimethylsiloxyethyl Dimethicone; Lauryl Polyglyceryl-3 Polydimethylsiloxyethyl Dimethicone.

Suitable W/Si emulsifying mixtures encompass Cyclopentasiloxane (and) PEG-10 Dimethicone (and) Disteardimonium Hectorite. (BENTONE® LUXE WS); Cyclopentasiloxane (and) Dimethicone/PEG-10/15 Crosspolymer (Andisil® CP 2050); Cyclopentasiloxane (and) Dimethicone (and) PEG/PPG-10/15 Dimethicone (and) Dimethicone/Vinyl dimethicone Crosspolymer (Cosmethicone® SF-6210); Cyclopentasiloxane (and) PEG/PPG-20/15 Dimethicone (SF 1528)

Further suitable emulsifiers for use according to the present invention are Glyceryl Hydroxystearate; Polyglyceryl-6 Distearate; Behenyl Behenate; Stearyl Behenate; Stearyl Stearate; Sorbitan Oleate; Sorbitan Laurate; Polysorbate 81; Hydrogenated Lecithin; Polysorbate 28; Sorbitan Oleate; Sorbitan Monopalmitate; Sorbitan Monooleate; Sorbitan Stearate; Sorbitan-Isostearate; Sorbitan Palmitate; Sorbitan sesquioleate; Laureth-9; PEG-20 Methyl Glucose Sesquistearate; PEG-200 Dilaurate; PEG 200 Dioleate; PEG 200 Distearate; PEG 200 Monolaurate; PEG 200 Monooleate; PEG-3 C12-18 alcohol; Steareth-2 PEG-2 stearyl ether; Calcium Stearoyl Lactylate; C12-C18 Diglycerides; Sucrose Stearate (and) Sucrose Distearate (NATPURE® SF).

The amount of the oily phase (i.e. the phase containing all oils and fats) present in compositions according to the present invention is preferably at least 10 wt.-%, such as in the range from 10 to 70 wt.-%, preferably in the range from 15 to 60 wt.-%, most preferably in the range from 20 to 50 wt.-%, based on the total weight of the composition.

According to one even more preferred embodiment, the compositions according to the present invention as outlined herein are W/O emulsions comprising an aqueous phase dispersed in an oily phase in the presence of at least one emulsifier of the group Lauryl PEG-10 Tris(Trimethylsiloxy)Silylethyl Dimethicone and/or Propylene Glycol Dicaprylate/Dicaprate; Stearalkonium Hectorite; Propylene Carbonate (MIGLYOL®-Gel 840 B) and/or Polyhydroxystearic Acid. The preparation of such W/O emulsions is well known to a person skilled in the art and illustrated in the examples.

Particularly preferred W/O emulsifiers to be used according to the present invention are polyglyceryl-2-dipolyhydroxystearate, polyglyceryl-3 diisostearate, cetyl PEG/PPG-10/1 dimethicone and PEG/PPG-18/18 dimethicone as well as mixtures thereof.

The compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 7 wt.-%, such as most in particular in the range of 1 to 5 wt.-%, based on the total weight of the composition.

Particular suitable co-surfactants are selected from the list of alkyl alcohols such as cetyl alcohol (Lorol C16, Lanette 16), cetearyl alcohol (Lanette O), stearyl alcohol (Lanette 18), behenyl alcohol (Lanette 22), polyhydroxystearic acid, glyceryl stearate, glyceryl myristate (Estol 3650), hydrogenated coco-glycerides (Lipocire Na10), Glyceryl Caprate, Glyceryl Dilaurate, Glyceryl Oleate, Glyceryl Stearate, Glyceryl monostearate, Sorbitan oleate, Apricot Kernel Oil Polyglyceryl-4 Esters and Stearalkonium Hectorite.

In all embodiments of the present invention, it is particular advantageous if the compositions according to the present invention also comprises Glyceryl Caprate, Glyceryl Dilaurate, Glyceryl Oleate, Glyceryl Stearate, Glyceryl monostearate, Sorbitan oleate and/or Stearalkonium Hectorite, most preferably Glyceryl Caprate, and/or Stearalkonium Hectorite.

Advantageous embodiments of the composition of the present invention also include those wherein the composition comprises one or more oils selected from butylene glycol dicaprylate/dicaprate, phenethyl benzoate, C₁₂-C₁₅ alkyl benzoate, dibutyl adipate, diisopropyl sebacate, dicaprylyl carbonate, di-C₁₂₋₁₃ alkyl tartrates, diethylhexyl syringylidene malonates, hydrogenated castor oil dimerates, triheptanoin, C₁₂₋₁₃ alkyl lactates, C₁₆₋₁₇ alkyl benzoates, propylheptyl caprylates, caprylic/capric triglycerides, diethylhexyl 2,6-naphthalates, octyldodecanol, ethylhexyl cocoates, phenethyl alcohol, propylene carbonate, ethylhexyl cocoate, C15-19 alkane, dimethicone, caprylyl methicone, cyclopentasiloxane, Hydrogenated Castor Oil, mineral oil, isoparaffin, isohexadecane, triisostearin. Preferably, the composition according to the present invention comprises as oil(s), propylene carbonate, ethylhexyl cocoate, C15-19 alkane, as well as mixtures thereof.

In a still further advantageous aspect of the invention, the compositions of the present invention further comprise a preservative and/or a preservative booster, preferably selected from the group consisting of ethanol, phenoxyethanol, sodium benzoate, potassium sorbate, phenethyl alcohol, hydroxyactophenone, ethylhexylglycerin, hexylglycerin, glyceryl caprylate, 1,3-propanediol, 1,2-pentanediol, 1,2-hexanediol, 1,2-octanediol (caprylyl glycol), 1,2-decanediol and 2-methyl-1,3-propanediol as well as mixtures thereof, most preferably selected from the group of phenoxyethanol and ethylhexyl glycerine as well as mixtures thereof. When present, the preservative respectively the preservative booster is preferably used in an amount of 0.01 to 5 wt. %, more preferably in an amount of 0.05 to 4 wt.-%, most preferably in an amount of 0.1 to 3.0 wt.-%, based on the total weight of the composition.

It is advantageous in accordance with the invention if the compositions according to the present invention further comprise one or more alkanediols from the group 1,3-propanediol, 1,2-pentanediol, 1,2-hexanediol, 1,2-octanediol, 1,2-decanediol and 2-methyl-1,3-propanediol. When present, the one or more alkanediols is preferably used in an amount of 0.1 to 7 wt. %, more preferably in an amount of 0.5 to 7 wt.-%, most preferably in an amount of 1.0 to 5.0 wt.-%, based on the total weight of the composition.

It is further advantageous in accordance with the invention if the composition of the invention comprises phenoxyethanol, ethylhexylglycerin, glyceryl caprylate and/or phenethyl alcohol, preferably phenoxyethanol and/or ethylhexylglycerin or glyceryl caprylate and/or phenethyl alcohol, most phenoxyethanol and ethylhexylglycerin or glyceryl caprylate and phenethyl alcohol

In another advantageous aspect, the compositions according to the present invention are free of any parabenes, benzethoniumchlorid, piroctone olamine, lauroylarginat, methylisothiazolinon, chlormethylisothiazolinon, bronopol, benzalkoniumchloride, formaldehyd releasing compounds, salicylic acid, triclosan, DMDM hydantoin, chlorphenesin and IPBC (Iodopropinylbutyl carbamate).

The compositions according to the invention may further contain one or more emollients which soothe and soften the skin. As an example, the emollient may be silicone (dimethicone, cyclomethicone), vegetable oils (grape seed, sesame seed, jojoba, etc.), butters (cocoa butter, shea butter), and petrolatum derivatives (petroleum jelly, mineral oil).

In another aspect, the compositions of the invention may comprise one or more fragrances selected from limonene, citral, linalool, alpha-isomethylionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic diester, cinnamal, amyl salicylate, alpha-amylcinnamaldehyde, alpha-methylionone, butylphenylmethylpropional, cinnamal, amylcinnamyl alcohol, anise alcohol, benzoin, benzyl alcohol, benzyl benzoate, benzyl cinnamate, benzyl salicylate, bergamot oil, bitter orange oil, butylphenylmethylpropional, cardamom oil, cedrol, cinnamal, cinnamyl alcohol, citronellyl methylcrotonate, citrus oil, coumarin, diethyl succinate, ethyllinalool, eugenol, Evernia furfuracea extract, Evernia prunastri extract, farnesol, guaiacwood oil, hexylcinnamal, hexyl salicylate, hydroxycitronellal, lavender oil, lemon oil, linalyl acetate, mandarin oil, menthyl PCA, methyl hexadecan, nutmeg oil, rosemary oil, sweet orange oil, terpineol, tonkabean oil, triethyl citrate, vanillin.

The compositions of the invention may advantageously comprise moisturizers. Moisturizers are compounds or mixtures of compounds which give cosmetic compositions the quality, after application to or distribution on the skin surface, of reducing the loss of moisture of the stratum corneum (also called transepidermal water loss (TEWL)) and/or of positively influencing the hydration of the stratum corneum.

Non-limiting examples of advantageous moisturizers for use in the compositions of the present invention include glycerol, lactic acid and/or lactates, especially sodium lactate, butylene glycol, propylene glycol, 1,3-propanediol, biosaccharide gum-1, Glycine soya, ethylhexyloxyglycerol, pyrrolidonecarboxylic acid, saccharide isomerate, panthenol and urea. Of further advantage in particular is the use of polymeric moisturizers from the group of the polysaccharides which are water-soluble and/or swellable in water and/or gellable with the aid of water. Especially advantageous, for example, are hyaluronic acid, chitosan and/or a fucose-rich polysaccharide which is registered in Chemical Abstracts under the registry number 178463-23-5 and is available, for example, under the Fucogel®1000 name from the company SOLABIA S.A. Moisturizers may also be used advantageously as active antiwrinkle ingredients for protection from changes to the skin of the kind occurring in skin aging, for example.

Particularly advantageous moisturizers to be used in the compositions according to the present invention are glycerin, sodium hyaluronate, saccharide isomerate and/or 1,3-propanediol, more preferably in a total amount selected in the range of 0.1 to 5 wt.-%, more preferably in the range from to 0.1 to 3 wt.-%, even more preferably in the range of 0.5 to 3 wt.-%, based on the total weight of the composition.

The compositions of the invention may further comprise advantageously, although not mandatorily, fillers which have the effect, for example, of further improving the sensorial and cosmetic properties of the formulations and evoking or intensifying a velvety or silky skin sensation, for example. Advantageous fillers in the sense of the present invention are starch and starch derivatives (such as tapioca starch, distarch phosphate, aluminum or sodium starch octenylsuccinate, and the like, for example), Valvance pigments which have neither primarily UV filter effect nor coloring effect (such as Valvance Touch 210 or 250 for example) and/or Aerosils® (CAS No. 7631-86-9) and/or talc and/or polyethylene, nylon, and silica dimethyl silylate.

The water phase of the compositions of the invention may advantageously comprise customary cosmetic auxiliaries, such as, for example, alcohols, particularly those of low C number, preferably ethanol and/or isopropanol, or polyols of low C number, and also ethers thereof, preferably propylene glycol, glycerol, electrolytes, self-tanning agents, and also, in particular, one or more thickeners, which may be advantageously selected from the group of silicon dioxide, aluminum silicates, polysaccharides and/or derivatives thereof, e.g., hyaluronic acid, xanthan gum, hydroxypropylmethylcellulose, particularly advantageously from the group of polyacrylates, preferably a polyacrylate from the group referred to as Carbopols, examples being carbopols of types 980, 981, 1382, 2984, and 5984, in each case individually or in combination. Further thickeners advantageous in accordance with the invention are those having the INCI designation Acrylates/C10-30 Alkyl Acrylate Crosspolymer (e.g., Pemulen TR 1, Pemulen TR 2, Carbopol 1328 from Lubrizol) and also Aristoflex AVC (INCI: Ammonium Acryloyldimethyltaurate/VP Copolymer) as well as Simugel NS (INCI: Hydroxyethyl Acrylate/Sodium Acryloyldimethyl Taurate Copolymer & Squalane & Polysorbate 60).

It is further preferred in accordance with the invention if the composition comprises a thickener. Suitable thickeners to be used according to the present invention encompass xanthan gum, crosslinked acrylate/C10-C30 alkyl acrylate polymer, hydroxyethyl acrylate/sodium acryloyldimethyl taurate copolymer, and/or vinylpyrrolidone/hexadecane copolymer, preferably xanthan gum and/or hydroxyethyl acrylate/sodium acryloyldimethyl Taurate, most preferably xanthan gum.

The amount of xanthan gum, if present is preferably selected in the range of 0.1 to 1 wt.-%, more preferably in the range of 0.15 to 0.75 wt.-%, most preferably in the range of 0.2 to 0.4 wt.-%, based on the total weight of the composition.

The oil phase of the compositions of the invention may advantageously also comprise customary cosmetic auxiliaries, such as, for example, thickeners for the oil phase such as Dextrin Palmitate (Rheopearl KL2, Chiba Flour Milling), Dextrin Palmitate and C15-19 Alkane (TILAMAR Fits, DSM), Polyurethane-79 and Caprylic/Capric Triglyceride (Oilkemia 5S, Lubrizol), Ethylene/Propylene/Styrene Copolymer and Butylene/Ethylene/Styrene Copolymer and Pentaerythrityl Tetra-di-t-butyl Hydroxyhydrocinnamate and Squalane (Veggi Gel, Aiglon), Dibutyl Lauroyl Glutamide and Dibutyl Ethylhexanoyl Glutamide and Octyldodecanol (AJK-OD246, Ajinomoto).

The amount of the thickener (e.g. the active thickening polymer), if present is preferably selected in the range of 0.1 to 15 wt.-%, more preferably in the range of 0.5 to 10 wt.-%, most preferably in the range of 1.0 to 8.0 wt.-%, based on the total weight of the composition.

It is further preferred in all embodiments of the present invention, if the compositions according to the invention comprise one or more organic UV-filter selected from the group consisting of homosalate, ethylhexyl salicylate, polysilicone-15, ethylhexyl methoxycinnamate, octocrylene, phenylbenzimidazole sulphonic acid or a salt thereof, bis-ethylhexyloxyphenol methoxyphenyl triazine, ethylhexyl triazone, diethylhexyl butamido triazone, methylene bisbenzotriazolyl tetramethylbutylphenol, tris-biphenyl triazine, butyl methoxydibenzoylmethane, diethylamino hydroxybenzoyl hexyl benzoate.

In another aspect, the composition may have an SPF of at least 15, preferably at least 20, most preferably of at least 30.

Advantageously in accordance with the invention, the composition of the invention comprises film formers. Film formers in the sense of the present invention are substances of various constitutions, and are characterized by the following properties: When a film former is dissolved in water or other suitable solvents, and when the solution is then applied to the skin, the film former, following evaporation of the solvent, forms a film which serves essentially to fix the UV-filters on the skin and thus to increase the water resistance of the product.

It is especially advantageous to select the film formers from the group of the polymers based on polyvinylpyrrolidone (PVP)

Particular preference is given to copolymers of vinylpyrrolidone, as for example the PVP hexadecane copolymer and the PVP eicosene copolymer, which are available under the trade names Antaron V216 and Antaron V220 from Ashland.

Likewise advantageous are further polymeric film formers, such as, for example, sodium polystyrene sulfonate, which is available under the trade name Flexan 130 from National Starch and Chemical Corp., and/or polyisobutene, available from Rewo under the trade name Rewopal PIB1000. Examples of further suitable polymers are polyacrylamides (Sepigel 305), polyvinyl alcohols, PVP, PVP/VA copolymers, polyglycols, acrylate/octylacrylamide copolymer (Dermacryl 79) Likewise advantageous is the use of hydrogenated castor oil dimer dilinoleate (CAS 646054-62-8, INCI Hydrogenated Castor Oil Dimer Dilinoleate), which can be acquired from Kokyu Alcohol Kogyo under the name Risocast DA-H, or else PPG-3 benzyl ether myristate (CAS 403517-45-3), which can be acquired under trade name Crodamol STS from Croda Chemicals.

The amount of the composition to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

The following examples are provided to further illustrate the compositions and effects of the present invention. These examples are illustrative only and are not intended to limit the scope of the invention in any way.

### EXAMPLES

### Short Description

The HMO's 3FL and 6′SL were evaluated for their SPF boosting effect. For this purpose various sunscreens were prepared according to standard methods in the art as outlined in table 1a and table 2a.

### In Vitro SPF Measurement

The in vitro SPF test was performed on PMMA plates (HD6 plates from Helioscreen, 5 cm×5 cm, roughness of Sa=6 μm): 32.5 mg of the respective formulation (i.e. 1.3 mg/cm²) were applied homogenously onto the PMMA plates and dried for 15 minutes.

The in vitro SPF was determined using a Labsphere 2000 UV Transmittance Analyzer: each PMMA plate was measured 9 times at different points on the plate resulting in 27 data points. The result is calculated as the average of these 27 data points.

TABLE 1a
Formulation 1 (W/O emulsion)

| | | | Conc. |
|---|---|---|---|
| Phase | Tradename | INCI name | [wt.-%] |
| A | Water Dem | Aqua | Ad 100 |
| A | Palmera G995V | Glycerin | 2.00 |
| A | Zemea ® Propanediol | Propanediol | 2.00 |
| A | Sodium Chloride Emprove | Sodium Chloride | 0.50 |
| | exp | | |
| A | RonaCare ® Zinc Sulfate | Zinc Sulfate | 1.00 |
| A | D-PANTHENOL | Panthenol | 0.50 |
| A | HMO | See table 1b | Table 1b |
| B | DOWSIL ES-5300 | Lauryl PEG-10 | 6.00 |
| | Formulation Aid | Tris(Trimethyl- | |
| | | siloxy)Silylethyl | |
| | | Dimethicone | |
| B | MIGLYOL ®-Gel 840 B | Propylene Glycol | 5.00 |
| | | Dicaprylate/Dicaprate; | |
| | | Stearalkonium Hectorite; | |
| | | Propylene Carbonate | |
| B | Palmester 1547 | Ethylhexyl Cocoate | 4.25 |
| B | Dermosoft ® GMC | Glyceryl Caprate | 0.50 |
| B | EMOGREEN L19 | C15-19 Alkane | 9.00 |
| B | Dowsil FZ-3196 Fluid | Caprylyl Methicone | 5.00 |
| B | Dispersun DSP-OL300 | Polyhydroxystearic Acid | 2.50 |
| B | dl-alpha-Tocopherol | Tocopherol | 0.20 |
| B | Aerosil 200 | Silica | 0.25 |
| C | PARSOL ® TX | Titanium Dioxide; Silica; | 7.00 |
| | | Dimethicone | |
| C | PARSOL ® ZX | Zinc Oxide; | 18.00 |
| | | Triethoxycaprylylsilane | |
| C | VALVANCE ® Touch 250 | Silica; Dimethicone | 2.50 |
| D | PEPHA ®-AGE | Senedesmus Rubescens | 1.00 |
| | | Extract; Aqua; | |
| | | Phenoxyethanol | |

TABLE 1b
Results

| | Conc. HMO | | |
|---|---|---|---|
| HMO | [wt.-%] | SPF in vitro | SPF boosting |
| Reference | — | 36.5 | — |
| Fucosyllactose (3FL) | 1 | 41.8 | +15% |
| Sialyllactose (6′SL) | 1 | 50.3 | +38% |

As shown in table 1b the HMO's 3FL and 6′SL boost the in vitro SPF between 15% and 38% in a W/O emulsion.

TABLE 2a
Formulation 2 (O/W emulsion)

| | | | Conc. |
|---|---|---|---|
| Phase | Tradename | INCI name | [wt.-%] |
| A | Water Dem | Aqua | Ad 100 |
| A | Palmera G995V | Glycerin | 5.0 |
| A | HMO | See table 2b | See table 2b |
| A | Keltrol CG-T | Xanthan Gum | 0.4 |
| B | Emulgade A6 | Ceteareth-6 Stearyl Alcohol | 2.5 |
| B | Emulgade B25 | Ceteareth-25 | 2.5 |
| B | Lanette O | Cetearyl alcohol | 4.0 |
| B | Lanette 22 | Behenyl Alcohol | 1.0 |
| B | Finsolv TN | C12-15 Alkyl Benzoate | 5.0 |
| B | Cetiol CC | Dicaprylyl Carbonate | 15 |
| B | Euxyl PE 9010 | Phenoxyethanol, | 1.0 |
| | | Ethylhexylglycerin | |
| B | Dispersun DSP- | Polyhydroxystearic acid | 0.5 |
| | OL300 | | |
| C | PARSOL ® TX | Titanium Dioxide; | 5.0 |
| | | Silica; Dimethicone | |
| C | PARSOL ® ZX | Zinc Oxide; | 10.0 |
| | | Triethoxycaprylylsilane | |

TABLE 2b
Results

| | Conc. HMO | | |
|---|---|---|---|
| HMO | [wt.-%] | SPF in vitro | SPF boosting |
| Reference | — | 9.23 | — |
| Fucosyllactose (3FL) | 1 | 11.45 | +24% |
| Sialyllactose (6′SL) | 1 | 12.62 | +37% |

As shown in table 2b the HMO's 3FL and 6′SL boost the in vitro SPF between 24% and 36% in an O/W emulsion.

### Comparative Data

TABLE 3a
(O/W emulsion)

| | | | 3a-I | 3a-II | 3a-III |
|---|---|---|---|---|---|
| Phase | Tradename | INCI name | Conc. [wt.-%] | | |
| A | Water Dem | Aqua | Ad 100 | Ad 100 | Ad 100 |
| A | Palmera G995V | Glycerin | 5.0 | 5.0 | 5.0 |
| A | HMO | See table 3b | See table 3b | | |
| A | Keltrol CG-T | Xanthan Gum | 0.4 | 0.4 | 0.4 |
| B | Emulgade A6 | Ceteareth-6 Stearyl | 2.5 | 2.5 | 2.5 |
| | | Alcohol | | | |
| B | Emulgade B25 | Ceteareth-25 | 2.5 | 2.5 | 2.5 |
| B | Lanette O | Cetearyl alcohol | 4.0 | 4.0 | 4.0 |
| B | Lanette 22 | Behenyl Alcohol | 1.0 | 1.0 | 1.0 |
| B | Finsolv TN | C12-15 Alkyl Benzoate | 5.0 | 5.0 | 5.0 |
| B | Cetiol CC | Dicaprylyl Carbonate | 15.0 | 15.0 | 15.0 |
| B | Euxyl PE 9010 | Phenoxyethanol, | 1.0 | 1.0 | 1.0 |
| | | Ethylhexylglycerin | | | |
| B | Dispersun DSP- | Polyhydroxystearic acid | 0.5 | 0.5 | 0.5 |
| | OL300 | | | | |
| C | PARSOL ® TX | Titanium Dioxide; Silica; | 5.0 | | |
| | | Dimethicone | | | |
| C | PARSOL ® ZX | Zinc Oxide; | | 10.0 | |
| | | Triethoxycaprylylsilane | | | |
| D | Parsol MAX | Methylene bis- | | | 16 |
| | | benzotriazolyl tetramethyl | | | |
| | | butylphenol (50% active) | | | |

TABLE 3b-I
Results - Formula 3a-I (one inorganic pigment, titanium dioxide)

| | Conc. HMO | | |
|---|---|---|---|
| HMO | [wt.-%] | SPF in vitro | SPF boosting |
| Reference | — | 3.63 | — |
| Fucosyllactose (3FL) | 1 | 3.66 | +0.8% |
| Sialyllactose (6′SL) | 1 | 3.67 | +1.1% |

TABLE 3b-II
Results - Formula 3a-II (one inorganic pigment, zinc oxide)

| | Conc. HMO | | |
|---|---|---|---|
| HMO | [wt.-%] | SPF in vitro | SPF boosting |
| Reference | — | 4.01 | — |
| Fucosyllactose (3FL) | 1 | 4.14 | +3.2% |
| Sialyllactose (6′SL) | 1 | 4.25 | +6% |

TABLE 3b-III
Results - Formula 3a-III (organic pigment)

| | Conc. HMO | | |
|---|---|---|---|
| HMO | [wt.-%] | SPF in vitro | SPF boosting |
| Reference | — | 6.44 | — |
| Fucosyllactose (3FL) | 1 | 6.72 | +4.3% |
| Sialyllactose (6′SL) | 1 | 4.25 | +4.8% |

As shown in table 3b-I to 3b-III no significant SPF boosting is observed when using solely one inorganic pigment respectively an organic pigment.

1. A composition comprising at least two different inorganic UV-filters and at least one human milk oligosaccharide, wherein the at least two different inorganic UV-filters comprises zinc oxide and/or titanium dioxide.

2. The composition according to claim 1, wherein the at least one human milk oligosaccharide is present in an amount effective to increase SPF of the composition.

3. The composition according to claim 1, wherein the amount of the at least one human milk oligosaccharide is from 0.2 to 5 wt.-%, based on the total weight of the composition.

4. The composition according to claim 1, wherein the at least one human milk oligosaccharide is selected from the group consisting of 2′-fucosyllactose, 3-fucosyllactose, difucosyllactose, lacto-N-fucopentaose I, 3′sialyllactose sodium salt, 6′sialyllactose sodium salt, lacto-N-neotetraose and lacto-N-tetraose; or a mixture thereof.

5. The composition according to claim 1, wherein the total amount of the inorganic UV-filters in the composition is in the range from 0.5 to 30 wt.-%, based on the total weight of the composition.

6. The composition according to claim 1, wherein the composition at least two different inorganic UV-filters comprises at least one titanium dioxide and at least one zinc oxide UV-filter.

7. The composition according to claim 1, wherein the least two different inorganic UV-filters are surface coated.

8. The composition according to claim 1, wherein the titanium dioxide comprises a double coated titanium dioxide having an inner silica and optionally an outer organic coating or silica coated titanium dioxide, uncoated zinc oxide and zinc oxide coated with triethoxycaprylylsilane.

9. The composition according to claim 1, wherein the composition comprises a carrier comprising at least 30 wt. % of water.

10. The composition according to claim 1, wherein the composition is a W/O emulsion.

11. The composition according to claim 1, wherein the composition further comprises at least one additional UV-filter selected from the group consisting of homosalate, ethylhexyl salicylate, polysilicone-15, ethylhexyl methoxycinnamate, octocrylene, phenylbenzimidazole sulphonic acid, a salt of phenylbenzimidazole sulphonic acid, bis-ethylhexyloxyphenol methoxyphenyl triazine, ethylhexyl triazone, diethylhexyl butamido triazone, methylene bisbenzotriazolyl tetramethylbutylphenol, tris-biphenyl triazine, butyl methoxydibenzoylmethane, and diethylamino hydroxybenzoyl hexyl benzoate; or a mixture thereof.

12. (canceled)

13. A method for increasing the sun protection properties (SPF) of a composition comprising at least two different inorganic UV-filters comprising adding at least one human milk oligosaccharide to the composition in an amount effective to increase the SPF of the composition, wherein the at least two different inorganic UV-filters comprises zinc oxide and/or titanium dioxide.

14. A method of preparing a composition comprising adding at least two different inorganic UV-filters and at least one human milk oligosaccharide to the composition during preparation of the composition, wherein the at least two different inorganic UV-filters comprises zinc oxide and/or titanium dioxide.

15. (canceled)

16. The composition according to claim 3, wherein the amount of the at least one human milk oligosaccharide is in the range from 0.5 to 4 wt.-%, based on the total weight of the composition.

17. The composition according to claim 4, wherein the at least one human milk oligosaccharide is selected from the group consisting of 2′-fucosyllactose, difucosyllactose, 3′sialyllactose sodium salt, 6′sialyllactose sodium salt, lacto-N-neotetraose and lacto-N-tetraose; or a mixture thereof.

18. The composition according to claim 5, wherein the total amount of the inorganic UV-filters in the composition is the range from 2 to 30 wt.-%, based on the total weight of the composition.

19. The composition according to claim 1, wherein the zinc oxide comprises uncoated zinc oxide or zinc oxide coated with triethoxycaprylylsilane.

20. The composition according to claim 9, wherein the composition comprises a carrier comprising 50 to 90 wt.-% of water.
